(19) United States
(12) Patent Application Publication
(10) Pub. No.: US 2008/0269755 A1
(43) Pub. Date: Oct. 30, 2008

(54) SYSTEM AND METHOD FOR LOCATING SAW BLADES AND LIKE CUTTING ACCESSORIES WITH A SURGICAL NAVIGATION SYSTEM

(76) Inventors: Donald W. Malackowski, Schoolcraft, MI (US); Jerry Culp, Oshtemo Township, MI (US); Jose Luis Moctezuma de la Barrera, Freiburg (DE)

Correspondence Address: INTEL. PROP./ RND;STRYKER CORPORATION, 4100 EAST MILHAM AVE., KALMAZOO, MI, 49001-6197, US

(21) Appl. No.: 12/116,587

(22) Filed: May 7, 2008

Related U.S. Application Data
(63) Continuation of application No. PCT/US2006/060649, filed on Nov. 5, 2006.
(60) Provisional application No. 60/735,063, filed on Nov. 9, 2005.

(51) Int. Cl. A61B 17/00

(52) U.S. Cl. 606/82

(57) ABSTRACT

A system and method for locating cutting accessories such as saw blades that, when actuated by the complementary driver, move relative to the driver. The saw blade is provided with one or more geometric features. The location of these features is identified by a pointer. The pointer is part of a surgical navigation system used to monitor the position of the driver to which the saw blade is attached. Based on the location of the geometric features, data regarding the head of the saw blade are generated. Thus, the surgical navigation has data indicating the position and orientation of the driver and the position of the head of the cutting accessory relative to the driver. Based on these data, the surgical navigation system generates data indicating the position and orientation of the head of the saw blade or like cutting accessory.

### RELATIONSHIP TO EARLIER FILED APPLICATION

This application is a continuation of International Application No. PCT/US2006/060649 filed 5 Nov. 2006 which claims priority under from U.S. Provisional Patent Application No. 60/735,063 filed 9 Nov. 2005. The contents of the above-listed applications are specifically explicitly incorporated herein by reference.

### FIELD OF THE INVENTION

This invention relates generally to saw blades and other planar shaped accessories used to perform surgical procedures. More particularly, this invention relates to a system and method for precisely locating such cutting accessories with a surgical navigation system.

### BACKGROUND OF THE INVENTION

A surgical navigation unit is sometimes used to aid the performance of a surgical procedure. Generally, a surgical navigation unit comprises one or more trackers, a localizer and a processor. The trackers are attached to the instruments and surgical tools employed to perform the procedure. The localizer receives signals from the trackers. Based on these signals, the localizer generates signals representative of the position and orientation of the trackers. The processor receives the signals generated by the localizer.

Preloaded into the processor is an electronic map of the site on or in the body of the patient at which the surgical procedure is being performed. The processor, based on signals received from the localizer, determines the position and orientation of the trackers. By extension, the processor also determines the positions and orientations of the surgical instruments and tools to which the trackers are attached. Based on these data, the processor determines the position and orientation of the surgical instruments and tools relative to the surgical site within the patient. The processor generates an image, presented on a display, which indicates the location of the surgical instrument or tool relative to the surgical site. A surgical navigation system thus functions as means to ensure a surgical instrument or tool is properly positioned relative to the surgical site.

Sometimes the surgical tool or instrument to which the tracker is attached is not the actual device physically applied to the surgical site. Instead, a device, sometimes referred to as a “cutting accessory,” is applied to the surgical site. For example, one surgical instrument (tool) employed to perform a surgical procedure is the powered handpiece. Internal to the handpiece is motor with a rotating shaft. Various types of cutting accessories such as drill bits or reamers are removably coupled to the handpiece shaft.

After a cutting accessory is attached to this type of handpiece, the distal end of the accessory, the tip of the drill or the forward end of the reamer head, is touched to a calibration unit. Prior to this step, data are stored in the processor containing the location of the calibration unit. The processor thus contains data from the tracker integral with the handpiece that indicates the position of the handpiece and data indicating the position of the calibration unit. By extension, from the touching of the distal end of the cutting accessory to the calibration unit, the processor determines the location of the distal end of the cutting accessory relative to the handpiece. Based on these data, the surgical navigation unit generates data that indicates the position of the cutting accessory as the handpiece to which the accessory is attached is moved. These data are used to generate data indicating the position of the distal end of the cutting accessory relative to the surgical site.

The above system works well for a cutting accessory that, on attachment to a handpiece, remains at a fixed location. Such accessories include rotating devices such as reamers or drills.

However, some cutting accessories shift position relative to the surgical tools to which they are attached. One such cutting accessory is the saw blade. Generally, a saw blade moves in an oscillating or reciprocating pattern. This enables the teeth, at the head of the blade, to cut through the tissue against which the blade is applied. Given that the blade head moves relative to the handpiece to which it is attached, it has proven difficult to simply touch the blade head to a calibration unit in order for the navigation system processor to generate data that indicates position of the blade head relative to the handpiece.

Furthermore, some handpieces include indexing mechanisms. The indexing mechanism shifts the angular orientation of the cutting accessory (saw blade) relative to an axis of the handpiece. This arrangement facilitates the positioning of the cutting accessory relative to the handpiece so it is in a position that allows the surgeon to perform the procedure with no or minimal ergonomic strain. Again, for a surgical navigation system to be able to determine the position of the blade head, it is necessary for the system to be provided with data indicating the indexed position of the blade.

A position sensor could be attached to the subassembly integral with the handpiece to which the blade is attached. Often, this subassembly is a blade mount. The sensor would generate a variable signal as a function of both the indexed position of the mount and its position in its reciprocal path of travel. Based on these sensor signals, the surgical navigation system processor could generate data that indicates the position of the blade head. However it is difficult to provide this type of sensor assembly. This difficult arises, in part, because this sensor assembly, like the other components of the handpiece, must be designed to withstand the rigors of autoclave sterilization (placed in a water vapor saturated environment at 270° F. at 30 psi (Gage)).

Thus, to date, it has proven difficult to provide a means for providing data to a surgical navigation system that indicates the position and orientation of the head of a cutting accessory, such as a saw blade, that engages in translational motion.

### SUMMARY OF THE INVENTION

This invention relates to exchangable surgical cutting accessories, such as saw blades. The cutting accessory of this invention is designed for integration into a surgical navigation system so that the navigation system generates data precisely indicating the position of the distal end of the accessory, the end applied to the surgical site.

The cutting accessory of this invention is formed with one or more reference features. The reference features are located at precisely known positions on the cutting accessory. For example, in a version of the invention when the cutting accessory is a saw blade, the features are located at known positions relative to the blade teeth.

The cutting accessory of this invention is used by attaching the accessory to the complementary surgical tool, handpiece, used to actuate the accessory. A pointer is placed on the reference features. Based on the position data generated by the pointer, a processor integral with the surgical navigation system derives data that indicates the position and orientation of the distal end of the cutting accessory relative to the complementary handpiece. Based on these data, the processor generates data indicating the location of the distal end of the cutting accessory relative to the surgical site.

It is still another aspect of this invention to provide the cutting accessory with reference features that are in a specific pattern unique to the specific type of the accessory. As a consequence of the pointer identifying these features, the surgical navigation system processor extracts data describing the specific patterns. Once the pattern data are determined, the system of this invention, by reference to look-up data, identifies the type of cutting accessory. Once this identification process is performed, the processor retrieves data used to regulate the operation of the handpiece so that handpiece operates in a manner based suited for actuating the cutting accessory. These data are then used to regulate handpiece operation.

### BRIEF DESCRIPTION OF THE DRAWINGS

The invention is pointed out with particularity claims. The above and further features of the invention are better understood from the following Detailed Description taken in conjunction with the accompanying drawings in which:

FIG. 1 depicts a surgical navigation system into which the cutting accessory identification system of this invention is incorporated;

FIG. 2 is perspective view of how a cutting accessory of this invention is attached to a surgical tool such as a powered handpiece;

FIG. 3 is a plan view of one cutting accessory, more particularly a saw blade, constructed in accordance with this invention;

FIGS. 4A and 4B are a flow chart of the process steps executed to determine the type of cutting accessory attached to the tool and how the distal end location of the cutting accessory is determined according to this invention;

FIG. 5 illustrates how a pointer is touched to a saw blade reference point according to this invention;

FIG. 6 is a diagrammatic illustration of the curve defined by the reference divots of the blade of FIG. 3;

FIG. 7 depicts the types of data stored in a blade definition file integrally associated with a particular blade of the invention;

FIG. 8 is a reproduction of image of a surgical navigation system of this invention indicating the location of the cutting accessory;

FIG. 9 is a flow chart of an alternative process of this invention how the system of this invention recognizes that a particular type of accessory has been fitted to a surgical tool;

FIG. 10 is a plan view of a second saw blade, constructed in accordance with this invention;

FIG. 11 is a flow chart of an additional alternative process of this invention to recognize the type of accessory fitted to a tool;

FIG. 12 is partial depiction of an alternative blade definition file;

FIG. 13 is a plan view of a third saw blade constructed in accordance with this invention;

FIG. 14 is a flow chart of an additional alternative process of this invention to recognize the type of accessory fitted to a tool; and

FIG. 15 is a diagrammatic illustration of the curve reconstructed by reference to the divots of the blade of FIG. 13.

### DETAILED DESCRIPTION

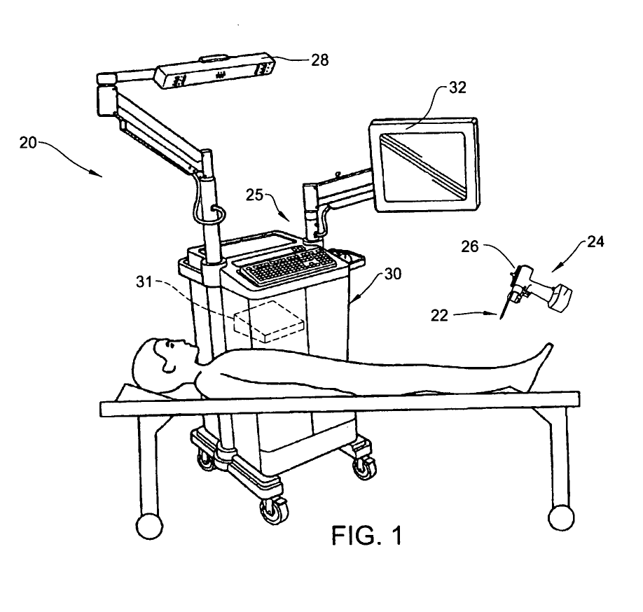

A surgical system 20 of this invention, including a cutting accessory 22, is shown in FIG. 1. System 20 includes a tool 24 used to position accessory 22 at a surgical site and actuate the accessory. In the described version of the invention, cutting accessory 22 is a saw blade and tool 24 is a handpiece that actuates the blade. The system 20 also includes a surgical navigation system 25. Surgical navigation system 25 includes a tracker 26 mounted to the tool 24. There is also a localizer 28.

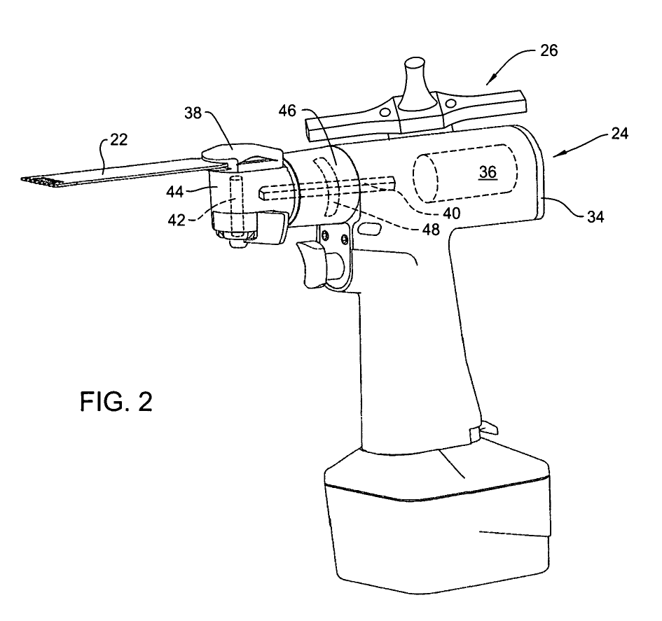

Tracker 26 actively or passively broadcasts a specific form of energy from one or more emitters or reflectors (resonators) (FIG. 2). Some trackers 26, for example, emit infra-red light or visible light. Other trackers emit RF or electromagnetic energy. Still other trackers emit ultrasonic energy. Localizer 28 monitors the position of the tracker 26. Specifically, the localizer 28 contains one or more receivers (not illustrated) capable of monitoring the energy emitted by the tracker 26. The receivers generate specific signals as a function of the direction from which the energy is transmitted. A processor 30, also part of surgical navigation system 25, receives the signals generated by the localizer receiver. Processor 30, based on the localizer receiver signals, generates signals representative of the position and orientation of the tracker 26 in free space.

A more detailed explanation of how a surgical navigation system operates is contained in the Applicant's incorporated by reference U.S. patent application Ser. No. 10/677,874, SURGERY SYSTEM, filed Oct. 2, 2003, U.S. Patent Publication No. U.S. 2004/0073279 A1, now U.S. Pat. No. ______. It should be appreciated that the system and method of this invention is not limited to versions of the invention using the above incorporated-by-reference navigation system. Other navigation systems may be incorporated into this invention. This includes navigations systems designed so that, based on signals transmitted by the localizer, components internal to the trackers generate signals representative of tracker position and orientation.

Tracker 26 is firmly attached to the tool 24. The geometry and dimensions of the tool 24 are known. Therefore, by extension, from the data indicating the position and orientation of the tracker 26, processor 30 generates data indicating the position and orientation of the tool 24. As discussed below, in this invention, processor 30 is also provided with data indicating the position and orientation of the cutting accessory 22 relative to the tool 24. Based on these data, data indicating the position and orientation of the position and orientation of the cutting accessory 22 as it is moved are generated.

Prior to the start of the surgical procedure, data defining the location of the site on or within the patient at which the surgical procedure is performed are loaded into the processor 30. More particularly, these data are stored in a memory integral with the processor 30 represented by phantom block 31. Based on these data and the processor-generated data indicating the position of the cutting accessory 22, processor 30 generates image-defining data that indicates the location of the cutting accessory 22 relative to the surgical site. These image defining data are used to present on a display 32 an image that indicates the location at the surgical site at which the cutting accessory 22 is located.

Simultaneously with the monitoring of the position of the tracker 26, localizer monitors the energy emitted by one or more marker, sometimes called fiducials 33 (one shown) affixed to the patient. Fiducials 33 include components that, like the members integral with the tracker 26, emit energy that can be sensed by localizer 28. The positions of the fiducials are tracked. Based on the data representative of fiducial position, processor 30 generates what is considered to be a dynamic reference frame for the body of the patient. The reference frame is considered “dynamic” because during the procedure, there may be some movement of the patient. Then, during the procedure, processor 30 maps this previously generated image-defining data of the patient's tissue onto the dynamic reference frame.

FIG. 2 illustrates a handpiece 24 to which a cutting accessory 22 and a tracker 26 are attached. Handpiece 24 includes a housing 34 in which a motor, represented by phantom cylinder 36, is mounted. Blade 22 is fitted in a blade mount 38 located distally forward of handpiece housing 34. (“Distal”, it shall be understood, means toward the surgical site to which the blade 22 and handpiece 24 are directed. “Proximal”, means away from the surgical site.) A drive mechanism, represented by a phantom bar 40, converts the rotary motion generated by the motor 34 into motion that reciprocates the blade mount 38 and, therefore, blade 22 in a back and forth pattern. A coupling assembly, represented in phantom by cylindrical rod 42, releasably holds the proximal end of the saw blade 22 to the blade mount 38.

Blade mount 38 is rotatably fitted in a head 44 located forward of handpiece housing 34. The head 44 is rotatably fitted in a collar 46 that extends forward from and is integral with the handpiece housing. A indexing mechanism, represented by phantom ring 48, releasably holds the handpiece head 44 in a fixed angular orientation around the longitudinal axis of the collar 46. This arrangement allows the angular orientation of the blade 22 to be set to an ergonomically convenient location in order to perform the intended surgical procedure.

The Applicant's Assignee's U.S. patent application Ser. No. 60/699,315, SURGICAL SAGITTAL SAW WITH QUICK RELEASE INDEXING HEAD AND LOW BLADE-SLAP COUPLING ASSEMBLY, filed 14 Jul. 2005 published as U.S. Pat. Pub. No. 2007/0016238 A1, now U.S. Pat. No. ______, the contents of which are incorporated herein, disclose a coupling assembly for holding the blade to the blade mount and an indexing assembly for selectively positioning the head. Both assemblies are understood to be examples of the types of assemblies that can be incorporated into the system 20 of the invention. This invention is not limited to systems including one or both of these assemblies.

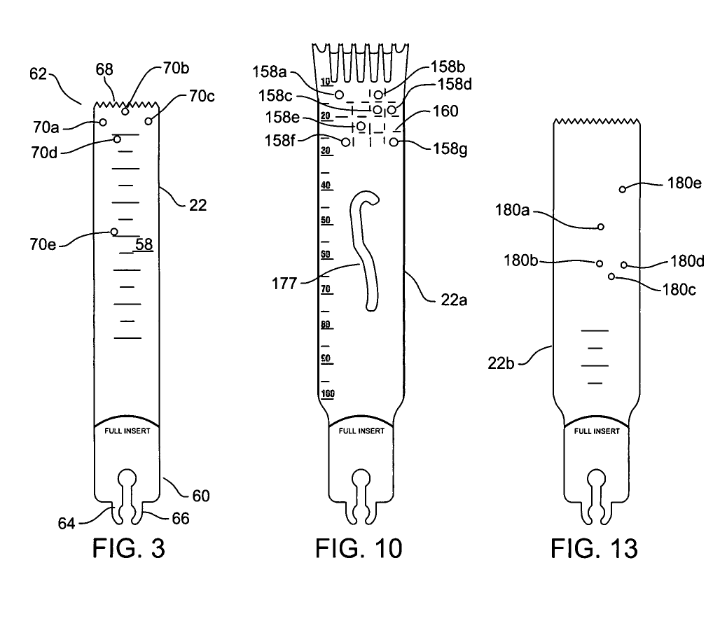

Blade 22, seen best in FIG. 3, includes opposed proximal and distal ends 60 and 62, respectively. Blade proximal end 60 is formed with coupling features 64, here represented by proximally extending tines 66. Coupling features 64 engage the coupling assembly integral with the handpiece 24. It should be appreciated that this invention is independent of the coupling features of the blade in which the invention is incorporated. The blade distal end 62 is formed with cutting teeth 68. The geometry of the blade teeth is not relevant to this invention. As discussed below, this invention provides data used to determine the geometry of blade teeth.

Saw blade 22 is further formed with reference features. In the blade of FIG. 3, these reference features are three divots 70a, 70b and 70c. Divots 70a-70c are formed on the blade so as to be a precisely known distance from teeth 68.

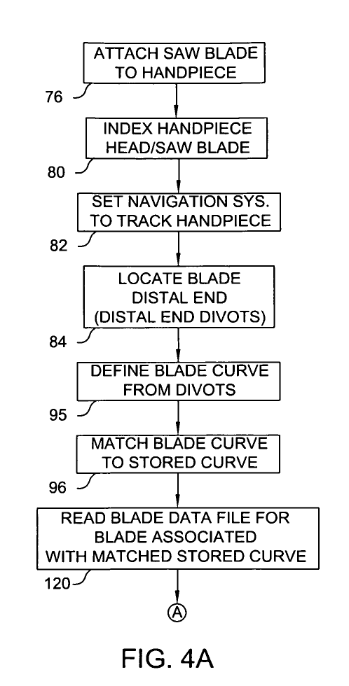

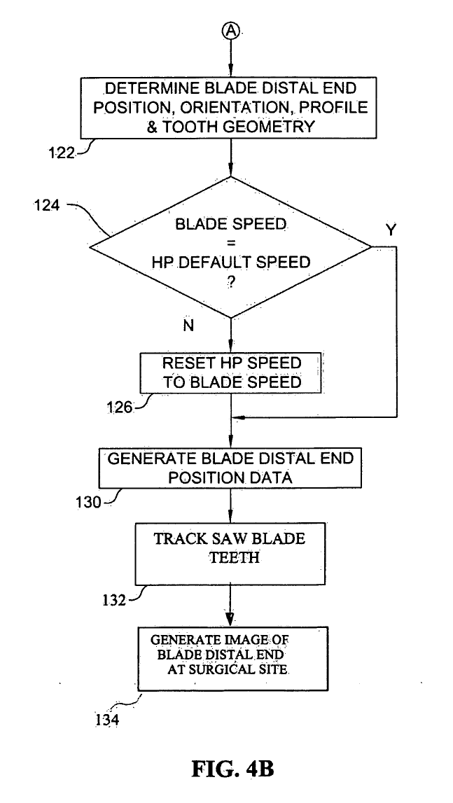

The handpiece with blade assembly of this invention for operation by first attaching the saw blade 22 to the handpiece 24, step 76 of FIG. 4A. In step 80, the handpiece indexing mechanism is set to place the blade mount and saw blade in the desired position to accomplish the surgical process. Also during the initial set up process, the appropriate steps are then executed to cause the surgical navigation system to track the tracker 26 and so that its processor 30 generates data indicating the position and orientation of the handpiece 24. In FIG. 4A this is represented by step 82. This step is shown as being executed after the blade is loaded and the indexing mechanism is set. This step may be executed earlier in the process.

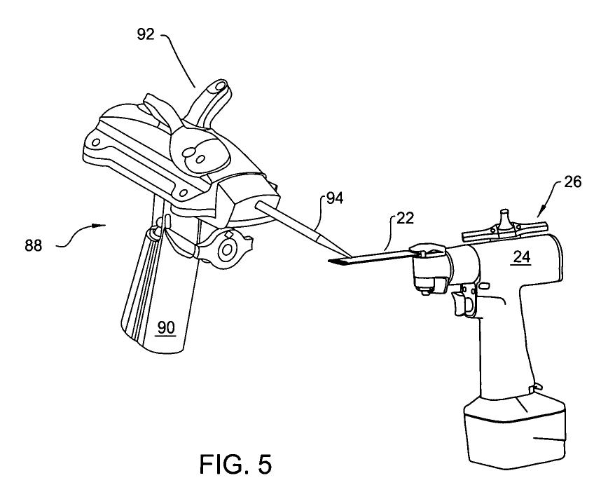

Once the surgical navigation system 30 starts tracking the handpiece 24, a step 84 is executed to locate the blade distal end 62. As seen by reference to FIG. 5, in step 84, a pointer 88 is touched to each of the divots 70a, 70b and 70c. Pointer 88 is a tool that is part of the surgical navigation system 25. The pointer 88 includes a handle 90 to which a tracker 92 is attached. (In the Figures, trackers 26 and 92 are aesthetically different.) An elongated tip 94 extends forward from the handle 90. The pointed end of the tip is the pointer components touched to the divots 70a-70c.

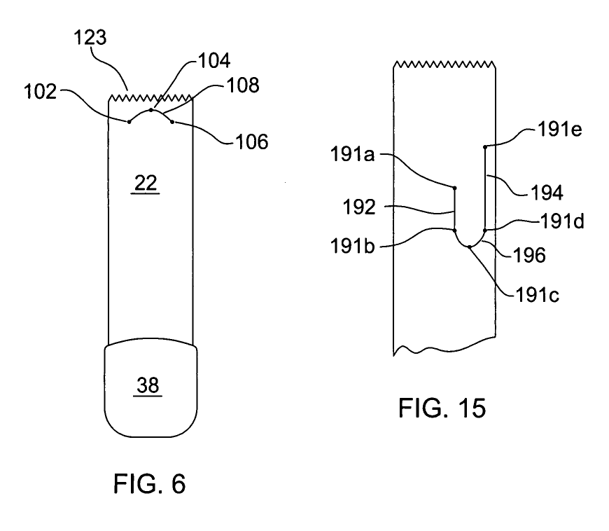

As the pointer tip 94 is touched to each recess 70a, 70b and 70c, localizer 28 monitors the signals emitted by the pointer tracker 92. Based on the signals emitted by the localizer, the surgical navigation unit processor 30 determines the position of each recess 70a-70c. In a step 95, processor 30 uses a curve construction algorithm to determine the shape and position of a curve defined by divots 70a-c. Examples of such curve construction algorithms that can be employed are curve fitting regression algorithms, polynomial curve fitting algorithms or curve fitting regression algorithms. Some curve construction algorithms require the knowledge of the three or more points (divots). However, some curve construction algorithms only require the knowledge of the location of two points (divots). For example, if the curve construction algorithm is based on there being an arc of predefined curvature, section of a circle, between the points, only the location of two divots are required. In still other versions of the invention, the “curve” is a set of line segments that form a spline. Again, these algorithms should be understood to be exemplary, not limiting. In FIG. 6, points 102, 104 and 106 represent the locations of divots 70a, 70b and 70c, respectively. Curve 108 is the curve generated in step 95 that is fit to points 102, 104, and 106.

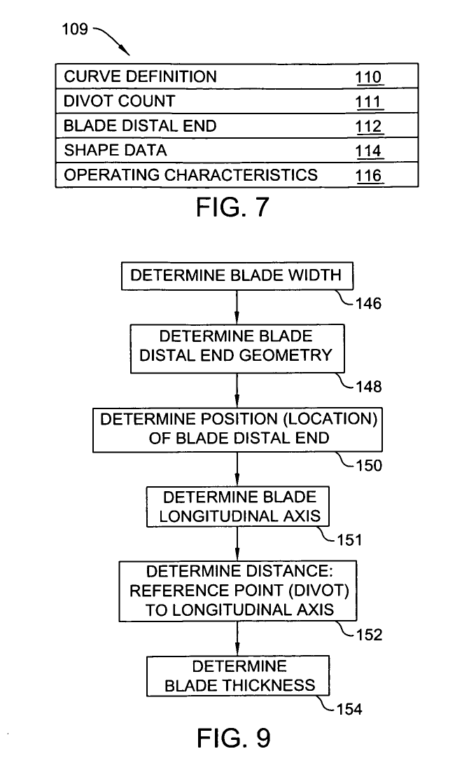

Once the curve 108 is defined, in a step 96, the processor determines to which one of a plurality of stored blade distal end curve geometries the curve defined in step 95 corresponds. These blade curve geometries are stored in the processor memory 31. Each stored blade curve geometry is associated with a blade definition file in the memory that is unique to a specific type of blade. Each file, one represented by block 109 of FIG. 7, contains a curve definition data field 110 with data that mathematically defines the curve integral with the blade 22. Curve definition field 110 includes a secondary divot count field 111. Divot count field 111 contains data indicating the number of divots that are used to define the curve.

Each blade definition file 109 also contains a data field 112 contains data indicating the distance from the defined curve to the actual teeth blade. A shape data field 114 contains data that define the actual geometry of the blade distal end 62. This geometry includes both the shape, perimeter profile of the blade distal end and the geometry of the teeth. Shape data field 114 also contains data indicating the thickness of the blade. An operating characteristics data field 116 contains data that define the operating characteristics of the blade. These characteristics include preferred, (default) blade speed and the maximum speed at which the blade should be actuated.

In step 96, a shape matching algorithm is used to match the curve 108 generated in step 95 to one of the stored curves from one of the fields 110. Suitable shape matching algorithms that can be employed are based on the radial distance from the center of mass (shape histogram), isotropic scaling or anisotropic scaling. This list should be understood as exemplary, not limiting. Additional matching of the blade to one of the blade definition files 109 is performed by matching the number of divots pointed to on the blade to define the curve with the number of divots in the divot count fields 111. Once the blade is matched with one of the blade definition files 109, in step 120, the rest of the data in the blade definition file 109 are read.

In a step 122, based on the position and orientation of the curve 108 and the data read from the selected blade definition file 109, processor 30 determines the position, shape and orientation of the blade distal end 62. Specifically, based on the data in the blade distal end data field 112, processor 30 determines how far forward of curve 108 the blade distal end is located. Based on the data in the shape data field 114, the processor determines that the shape of the blade distal end and the tooth geometry. In FIG. 6 this is represented by the saw tooth plot 123 located forward of curve 108. Thus, this particular blade has a slightly curved distal end with saw tooth teeth.

In a step 124, the system of this invention also determines if the operating speed of the handpiece can be reset. Generally a handpiece is set so that the motor causes the blade mount to oscillate at a rate equal to the slowest oscillation rate of any of the potential blades that may be fitted to the handpiece 24. Typically, this rate is between 10,000 and 15,000 cycles per minute. (A “cycle” is one complete back and forth oscillation of the blade mount 38.) Some blades can be operated at higher cycle rates. Thus, in step 124 the processor 30 determines if the data field 116 for the blade indicates the blade can be operated at a rate greater than the low cycle rate default rate and what the default rate is for that blade.

If, in step 124 it is determined the blade can be operated at a speed greater than the default low cycle rate setting, processor 30 resets the operating speed of the motor, step 126. The Applicants' Assignees' U.S. patent application Ser. No. 60/694,592, filed 28 Jun. 2005, Powered Surgical Tool With Sealed Control Module, incorporated herein by reference, discloses one such assembly for resetting the operating speed of the motor. Alternatively, if in step 124 the data from field 116 indicates the cycle rate is the lowest cycle rate, step 126 is bypassed.

As a consequence of the execution of steps 82 and 122, processor 30 contains data indicating the position of the handpiece 24 and the location of the blade distal end 62 (saw teeth). Based on these data, processor 30, in step 130, generates data indicating the distance from the handpiece 20 to the teeth 68. Specifically, in step 130, the processor 30 generates data indicating the index orientation of the blade 22. Processor 30, in step 130, also generates data that indicates the position of the blade distal end 62 relative to a fixed reference of the blade mount. Diagrammatically in FIG. 6, this is seen by the positioning of tooth plot 123 a specific distance forward of the blade mount 38.

Once this final step is performed, system 20 of this invention is ready for use. As a consequence of the tracking of handpiece 24 and the known location of the blade distal end relative to the handpiece, the position and orientation of the distal end teeth 68 of the saw blade are known. Because the saw blade oscillates back and forth at a relatively high speed, appx 10,000 to 20,000 cycles per minute, the processor does not generate data indicating the instantaneous position of the blade 22. Instead, in step 134, processor 30 generates an image of the blade 106 that represents the whole of the path of movement of the blade.

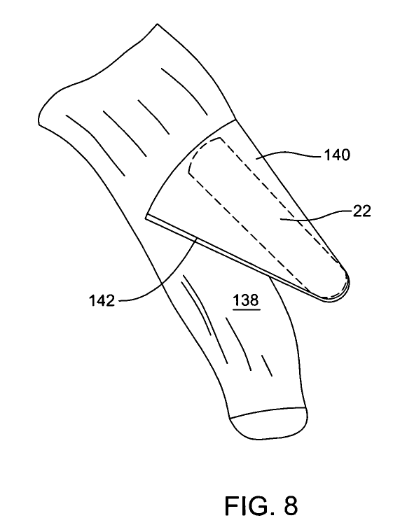

Consequently, as represented by FIG. 8, surgical personal viewing the surgical site on display 32 see the whole of the space subtended by the blade as a consequence of its actuation as subtending the adjacent tissue. In this Figure, a bone 138, such as femur, is shown as being the body tissue on which the surgical procedure is being performed. Arcuate slice 140 is the image presented on the display to illustrate the space within which the blade is oscillating. Within slice 140, shown for reference only and not part of the generated image, blade 22 is depicted as being a phantom element within slice 140.

It should further be appreciated that, as part of the generation of the image, in step 134, processor 30 generates data that represents the thickness of the blade 22 and the space subtended by the blade. These data are based on the data retrieved from the shape data field 114. In FIG. 8, this is represented by the lateral side wall 142 of slice 140. The generated image thus provides surgical personal a means to readily determine the extent to which the blade teeth are being applied against the tissue to which they should be applied and if the blade is encroaching tissue against which it should not be applied.

Thus, the system of this invention provides surgical personnel with a means to view at a surgical site with a surgical navigation system a saw blade or like cutting accessory that shifts position relative to the handpiece to which it is attached.

The system and method of this invention may employ other means to, from the divot positions, derive data defining the distal end geometry of the saw blade.

In one such alternative method. The curve defining divots 70a-c are located at precise distances from edge surfaces that define the blade distal end 62. Specifically, divots 70a and 70c, the divots that define the opposed ends of the curve, are each located defined distances from the side edges of the blade distal end. Each of the divots, 70a, 70b and 70c, are located a set distance proximally rearward from most distal tips of the blade teeth 68. Each of these distances is constant for all blades 22 used in the system, regardless of blade length, width or shape.

In this structure of the invention, there are fourth and fifth divot 70d and 70e, respectively. Divot 70d is located rearwardly of divots 70a-70c. Divot 70d is further formed on the face of the saw blade 22 to be located a defined distance from the line defined by divots 70a and 70c. Divot 70e is formed on the blade so as to be offset a specific distance from the longitudinal axis that extends through the blade.

The process steps executed to determine blade geometry in this embodiment of the invention are now described by reference to the flow chart of FIG. 9. More particularly, the flow chart of FIG. 9 illustrates the steps performed after step 84 is executed. In this version of the invention, during the execution of step 84, pointer 88 is touched to all four divots 70a-70d to determine their individual positions.

Once the positions of divots 70a-70d are determined, in a step 146 the width of the blade is determined. This width is determined by first determining the distance between the two opposed outermost divots. In the present example, these are divots 70a and 70c. Once this distance is determined, processor 30 determines the width of the blade according to the following formula:

Blade Width=Distance Between Outermost Curve Defining Divots+K (1)

Here, K is constant stored in the processor memory 31. Constant K is the same for all blades.

Distal end blade geometry is then determined in a step 148. In step 148 curve fitting algorithm that may be one of algorithms employed in step 95 is used to establish the curve defined by divots 70a, 70b and 70c. The curve generated in step 148, which may be a line, is the geometry of the distal end perimeter of the blade 22.

In a step 150, processor 30 determines the position of the blade distal end 62. This determination is made according to the following formula:

Blade Distal End Position=Curve Position+W (2)

Here, W is a constant also stored in processor memory 31. Constant W is identical for all blades.

Alternatively, in step 150, processor 30 determines the distance D between divot 70b and the line defined by divots 70a and 70c. Then in step 144, the blade distal end position is determined by one of the following formulas:

Position of the Line Defined By Divots 70a and 70c+Distance Between The Line and Divot 70b+W (Constant)=Blade Distal End Position (2a)

or

Position of the Line Defined By Divots 70a and 70c+(Distance Between The Line and Divot 70b) B=Blade Distal End Position (2b)

Here B is a constant coefficient stored in memory 31. Constant coefficient B is identical for all blades.

Determination of blade thickness starts with mathematical generation of position of the longitudinal axis of the blade, step 151. This step is determined by first calculating the midpoint of the straight line between divots 70a and 70c. This point, along with the midpoint of the curve calculated in step 145, are considered to be two points of the longitudinal axis of the blade. Alternatively, the second point is the position of divot 70b. The mathematical definition of this axis points is generated. In a step 152, the distance from divot 70e to the longitudinal axis is determined.

In a step 150, blade thickness is then determined according to the following formula:

Blade Thickness=Z (Distance From Long. Axis To Divot 70e) (3)

Here Z is a constant coefficient also stored in processor memory 31. Constant coefficient Z is constant for all blades.

Once blade distal end shape, length, position and thickness are determined, the surgical navigation system of this invention is ready to generate data indicating the position of the blade. These data are then used to produce images of the presence of the blade relative to the surgical site. These data and images are generated using the same methods discussed above with respect to steps 130 and 134 of the first described method of this invention.

An advantage of the above system and method of this invention is that it eliminates the need to store in processor memory 31 a number of different blade definition files. Also this version of the invention eliminates the need to match a reconstructed or generated shape formed on the blade with one of a number of stored shapes.

FIG. 10 illustrates a blade 22a with alternative divot pattern from which blade position and profile are determined. Here, blade 22a has a series of divots 158a-g. Each divot 158a-g is disposed in one of 16 locations within a 4×4 grid 160 shown in phantom. In some versions of the invention, divots 158a and 158g are, respectively always located in diagonally opposed locations at corners of the grid 160. Divots 158a and 158g thus serve as reference divots from which the position of the other divots on the grid 160 are determined. Divots 158b-f are located in other locations on the grid 160 as a function of blade type.

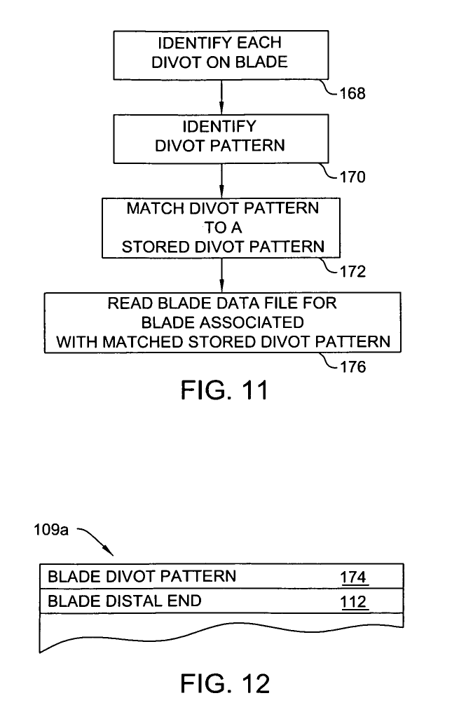

In an equivalent to step 84, step 168 of FIG. 11, pointer 88 is used to identify the location of each divot. Then, in step 170, based on the measured position of each divot, the divot pattern is reproduced by the navigation processor 30. In some versions of the invention, this pattern is reproduced based on the position of the divots having “known” locations, divots 158a and 158g. In a step 172, this profile is matched to one of a number of stored profiles in number of blade definition files 109a, one partial shown in FIG. 12. Files 109a are substantially identical to the previously described blade definition files 109. However instead of a curve profile data field for the associated blade, the file contains a blade divot pattern field 174. The divot match pattern field contains data describing the divot pattern that is unique to the blade type.

In step 172, a matching process is used to match the divot pattern of the attached blades to one of the stored divot patterns. Processor 30 interprets the match as indicating the blade associated with the matched pattern is attached to the handpiece. Consequently, a step 176, identical to step 120, is executed to read the remainder of the data describing the characteristics of the matched blade into the processor 30. The operation of the saw 24 is controlled as before.

In some versions of this invention, the data in the blade distal end field 112 indicates the distance from the forward most row of grid 160 at which the blade distal end is located.

In still another alternative version of the invention, instead of divots, an elongated groove 177 is formed on the flat of the blade 22a as seen in FIG. 10. In this version of the invention, after the blade 22a is mounted to the handpiece 24 and the handpiece head 44 is indexed, in a step similar to step 84 the position and orientation of the groove 177 is obtained. These data are obtained by placing the tip of the pointer 88 in the groove and running the tip along the whole of the length of the groove.

Then, in a step similar to step 96, the shape matching algorithm is used to match the shape of groove 177 to a corresponding shape in one of the blade characteristic files. Thus, as a result of the execution of this step, processor 30 matches the blade to the associated stored blade definition file 109. The previously described steps employed to read the data describing the blade and to operate the saw 24 are executed as before.

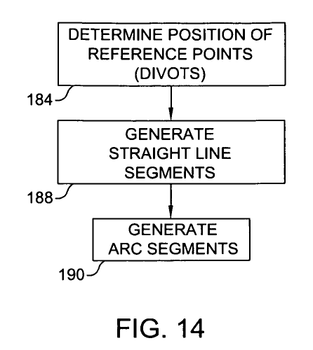

In still another version of the invention, a pattern reconstruction process is employed to determine basic information obtained from a set of divots or other reference points formed on a blade. This method may be employed on a blade such as blade 22b depicted by FIG. 13. Here, blade 22b is provided with divots 180a-e. In a step 184, shown in the flow chart of FIG. 14, pointer 88 is employed to determine the position of the divots.

Pattern reconstruction starts with generation of the straight line segments of the pattern, step 188. Specifically, processor 130 determines the distance between divots that area spaced apart a distance greater than a distance m. In FIG. 15, the two line segments 192 and 194 are shown. In FIG. 15, divots 180a-e are represented as points 191a-e.

In a step 190 processor 30 generates an arc between the three divots that are spaced apart a distance less than n. In FIG. 15, this curved portion of the pattern is represented by arc 196.

Data defining the characteristics of blade 22b are then obtained based on the reconstructed pattern. In some versions of the invention, the pattern matching algorithm is employed to match the reconstructed pattern to one of the curves, patterns, in a blade definition file 109; a step similar to step 96 is executed.

Alternatively, each segment of the pattern of the pattern is recognized as being proportional to or associated with a specific blade definition characteristic. For example, in some versions of the invention line segment 192 is proportional to (by a known coefficient or constant) the distance from the proximal end of arc 196 to the distal end of the blade 22b. Arc 196 has a unique relationship with a specific distal end blade curvature. Line segment 194 is proportional to the blade thickness. Thus, steps similar to steps 140, 142 and 144 are executed to, based on the segments forming the reconstructed pattern, generate data describing the characteristics of the blade 22b.

Alternative versions of the system of this invention are possible. For example, in some versions of the invention, a protective cap may be fitted over the blade. In these versions of the invention, the divots or blade type-defining plots are formed on the cap. In these versions of the invention, it should be clear that the cap is not removed until after the blade is attached to the handpiece and the pointer is used to identify the divots or other blade type-defining plot.

Likewise, it should be clear that the blade type-defining indentations need not always be recessed features formed on the blade flat. In alternative versions of the invention, these features may simply be markings printed or otherwise formed on the blade. In some versions of the invention, these features are raised relative to the flat surfaces of the blade.

In some versions of the invention, these blade defining features may not even be formed or located on the blade flat. For example, in some versions of the invention, the blade-defining features may be notches that extend inwardly from the blade longitudinal side edges. The number of notches, notch lengths and/or inter-notch spacing is unique to each blade type.

When the invention is practiced with the a cutting accessory attached to the described surgical saw, the components internal to the saw typically have such a high cumulative quantity of static friction that, between successive taping off of the pointer 88 to successive divots 70a, 70b and 70c, the saw blade remains static. However, some tools 24 with which this invention may be practiced are not similarly designed. There is a possibility with that between individual divot tap offs, the cutting accessory 22 could move. When the invention is practiced with this type of tool and cutting accessory assembly, it may be necessary to first place the tool and/or cutting accessory in jig that holds the cutting accessory static. This assures that since the cutting accessory is immobilized, the divot location determinations made as a result of the successive tap offs accurately represent the relative positions of the divots.

In some versions of the invention, the data defining the characteristics of the blade may be stored in an RFID or other machine readable memory unit integral with the blade. Thus the RFID or other device is contained in a plastic block. If the memory device is an RFID, the coil through which signals are inductively exchanged with the RFD is also embedded in a block. The block is contained in a window formed in the blade. The saw is formed with a complementary reader.

Alternatively, the RFID or other memory device is attached to the cap fitted over the blade or be contained within the blade packaging.

Therefore, it is an object of the appended claims to cover all such variations and modifications that come within the true spirit and scope of this invention.

What is claimed:

1. A surgical saw blade, said saw blade including:
a blade body;
cutting teeth that extend from the blade body;
coupling features integral with the blade body that releaseably engage a surgical saw blade coupling assembly so that the blade body is secured to and actuated by a surgical saw and
at least one geometric feature on said blade body the position of which is detectable by a surgical navigation pointer wherein the position of said at least geometric feature on said blade body is a function of at least one selected from the group comprising: the location of the cutting teeth; the profile of the cutting teeth; the thickness of the blade; the width of the blade; the length of the blade; a speed at which the blade can be actuated.

2. The surgical saw blade of claim 1, wherein said blade body is formed with a plurality of said geometric features that are spaced apart from each other, wherein the relative locations of said geometric features to each other is a function of at least one selected from the group comprising: the location of the cutting teeth; the profile of the cutting teeth; the thickness of the blade; the width of the blade; the length of the blade; a speed at which the blade can be actuated.

3. The surgical saw blade of claim 1, wherein said at least one geometric feature is an elongated slot, wherein said slot has a shape and a size that is a function of at least one selected the location of the cutting teeth; the profile of the cutting teeth; the thickness of the blade; the width of the blade; the length of the blade; from the group comprising: a speed at which the blade can be actuated.

4. The surgical saw blade of claim 1, wherein:
the blade body has a planar surface; and
said at least geometric feature is a divot or slot formed in the blade body planar surface.

5. A surgical tool system, said system comprising:
a surgical saw having a motor and a coupling assembly for releasably coupling a saw blade to the motor so that the saw blade will be actuated by said motor;
a surgical navigation unit for tracking the location of the surgical saw;
a surgical saw blade, the saw blade having: a body; cutting teeth that extend from the body; and coupling features for cooperating with the saw coupling assembly to releasably couple the saw blade body to the motor, wherein said blade is provided with at least one geometric feature the location of which can be located by the surgical navigation unit; and
a pointer associated with said surgical navigation unit for locating the saw blade geometric feature, wherein
said surgical navigation unit is configured to:
based on the location of the saw blade at least one geometric feature determines at least one from the group of the location of the cutting teeth; the profile of the cutting teeth; the thickness of the blade; the width of the blade; and the length of the blade and,
based on the determination made by the surgical navigation unit and the tracked location of the surgical saw, the surgical navigation unit generates data indicating the location of the saw blade

6. The surgical tool system of claim 5, wherein:
the saw blade blade body is formed with a plurality of said geometric features that are spaced apart from each other, wherein the relative locations of said geometric features to each other is a function of at least one selected from the group comprising: the location of the cutting teeth and
based on the relative locations of said saw blade geometric features to each other, the surgical navigation unit: determines the position of the cutting teeth relative to the surgical saw; and based on the position of the cutting teeth and the tracked location of the surgical saw, the surgical navigation unit generates data indicating the location of the saw blade cutting teeth.

7. The surgical tool system of claim 5, wherein:
the saw blade blade body geometric feature that has a distinct geometry and shape that is a function of the location of the cutting teeth;
the surgical navigation unit is further configured to determine the geometry and/or shape of the saw blade geometric feature and; based on the geometry and/or shape of the geometric feature, determine the position of the saw blade teeth relative to the surgical saw; and based on the position of the saw blade teeth and the position of the surgical saw, determine the location of the saw blade cutting teeth.

8. The surgical tool system of claim 5, wherein:
said saw blade body geometric feature has shape that is unique to a set of data that defines the characteristics of the saw blade:
said pointer determines the shape of the saw blade geometric feature for the surgical navigation unit;
based on the shape of the saw blade geometric feature, the surgical navigation unit matches the saw blade to the appropriate set of data and reads the data; and
based on the read data, the surgical navigation unit determines the position of the saw blade cutting teeth.

9. The surgical tool system of claim 8, wherein the saw blade geometric feature shape is a curve or a multi-segment shape that includes straight and arcuate sections.

10. The surgical tool system of claim 8, wherein the saw blade geometric feature is a set of divots, said divots having a geometric relationship to each other that is unique to a specific set of saw blade data

11. The surgical tool system of claim 5 wherein:
the saw blade body geometric feature is located on the saw blade so as to be a defined distance from the saw blade teeth; and
the surgical navigation unit is configured to determine the position of the saw blade teeth relative to the surgical saw by calculating a position for the saw blade teeth as a function of the location of the saw blade body geometric feature.

12. The surgical tool system of claim 5, wherein:
the saw blade geometric feature corresponds to data indicating a speed at which the saw blade should operate;
the surgical navigation unit, based on the determination of the location of the saw blade geometric feature generates data indicating the speed at which the should be operated

13. A method for determining the position of the teeth of a surgical saw blade, the saw blade being actuated by a powered handpiece, said method including the steps of:
releasably coupling a saw blade to a handpiece, the saw blade having: teeth; and a geometric feature the position of which can be located, the geometric feature having a shape that are related to the position of the saw blade teeth and the handpiece having a motor for actuating the saw blade;
with a surgical navigation system, tracking the position of the handpiece,
with the surgical navigation system, determining the location and shape of a geometric feature on the saw blade;
based on the location or shape of the saw blade geometric feature, determining the location of the location of the saw blade teeth relative to the handpiece; and
based on the position of the handpiece and the location of the saw blade teeth relative to the handpiece, generating data indicating the position of the saw blade teeth.

14. The method of determining the position of the teeth of a saw blade of claim 13, wherein said step of determining the location of the saw blade teeth is performed by:
matching the location or shape of the saw blade geometric feature to one from plurality of unique data sets, each data set including data describing the characteristics of the saw blade;
reading the matched data set; and
based on the data read from the matched data set, determining the location of the saw blade teeth.

15. The method of determining the position of the teeth of a saw blade of claim 14, further including the step of, based on the data read from the selected data set, determining an operating speed for the handpiece motor.

16. The method of determining the position of the teeth of a saw blade of claim 14, wherein:
the saw blade has a plurality of spaced apart geometric features;
in said geometric feature location and shape determining step, the location of the geometric features relative to each other is determined;
based on the location of the geometric features relative to each other, a specific pattern for the geometric features is determined,
in said location or shape matching step, the determined pattern of the geometric features is matched to a pattern that is specific to one data set to determine the matched data set.

17. The method of determining the position of the teeth of a saw blade of claim 13, wherein:
said step of determining the position of the saw blade teeth is performed generating data indicating the position of the saw blade teeth by calculating a position for the saw blade teeth as a function of the location of the saw blade geometric feature.
